(19) United States
(12) Patent Application Publication
(10) Pub. No.: US 2026/0104414 A1
(43) Pub. Date: Apr. 16, 2026

(54) CHEMILUMINESCENCE-BASED DUAL FLOW RAPID DIAGNOSTIC TESTING PLATFORM AND METHODS OF USE

(71) Applicant: University of Cincinnati, Cincinnati, OH (US)

(72) Inventors: Chong H. Ahn, Cincinnati, OH (US); Heeyeong Jang, Cincinnati, OH (US); Supreeth Setty, Cincinnati, OH (US)

(73) Assignee: University of Cincinnati, Cincinnati, OH (US)

(21) Appl. No.: 19/355,295

(22) Filed: Oct. 10, 2025

Related U.S. Application Data
(60) Provisional application No. 63/706,340, filed on Oct. 11, 2024.

(51) Int. Cl. G01N 33/543; C12Q 1/28; G01N 21/76; G01N 33/68

(52) U.S. Cl. CPC G01N 33/54388; C12Q 1/28; G01N 21/76; G01N 33/6887; G01N 2333/4712; G01N 2333/908

(57) ABSTRACT

A lateral flow assay for detecting a target in a liquid sample is disclosed. The lateral flow assay may comprise a first flow path and a second flow path for the liquid sample that merge at or before a membrane that generates a signal of immunoassay. Also disclosed are cartridges that include the lateral flow assay and methods for detecting a target in a liquid sample using the lateral flow assay.

### CROSS-REFERENCE TO RELATED APPLICATIONS

The present application claims priority to U.S. Provisional Application No. 63/706,340, filed Oct. 11, 2024, the entirety of which is incorporated herein by reference.

### TECHNICAL FIELD

This disclosure relates to the field of rapid diagnostic testing. Specifically, the disclosure pertains to chemiluminescence-based lateral flow assays.

### BACKGROUND

Recently, there has been an increasing demand for the development of new rapid diagnostic testing (RDT) platforms capable of performing highly sensitive assays for early screening and diagnosis of chronic or infectious diseases. Although currently available RDT platforms can perform the lateral flow assays (LFAs) desired for the most rapid and qualitative diagnostics at a low cost, they are unable to achieve an ultra-high sensitivity, critically desired for specific life-threatening diseases, such as acute myocardial infarction (AMI). Moreover, evidence of increased cardiovascular disease rates after the infection and vaccination of coronavirus diseases (COVID) has been reported, leading to an increasing demand for developing new diagnostic platforms for AMI. Early detection of cardiac troponin I (cTnI) is critical to save patients with AMI, so the assay of cTnI, which is able to achieve the desired high-sensitivity, is considered as one of the most challenging issues in most strip-based RDT platforms.

Typical LFA strips comprise a single layer of multiple, porous membrane pads such as a sample pad, conjugate pad, nitrocellulose (NC) membrane, and absorbent pad. The hydrophilic surface of porous membranes facilitates the continuous flow of the fluid from the sample pad to the absorbent pad. In the LFA-based RDT, color differences emitted from the test and control lines are mainly due to the accumulation of gold nanoparticles (AuNPs), which is proportional to the number of formed immunocomplexes. Although the AuNPs facilitate colorimetric detection, the lower sensitivity of such LFA strips is attributed to their poor response in low concentration, which makes the LFA strips unattractive for diagnosing low-abundant biomarkers.

The signal from the antigen-antibody complex can be highly amplified by using detection antibody (DAb) labeled with enzyme, which is known as enzyme-linked immunosorbent assay (ELISA). In the ELISA, the products generated from the multiple iterations of the substrate-enzyme-product process allow the assay to achieve an enhanced limit of detection (LoD). Among the available ELISAs, the chemiluminescence (CL)-based ELISA is considered as one of the most sensitive and suitable detection assays to be adoptable for LFA strips, since a sensitivity 10˜100 times higher than that of colorimetric detection can be achieved. The major advantages of the CL detection method over others are a lower background signal and simple implementation using photodetectors to measure the output signal without an excitation light source.

A challenge in adopting the CL-based ELISA on LFA strips arises from the lack of methods for storing liquid CL substrates and realizing the desired sequential dual flows on the LFA strip at the same time. For example, a sample such as serum should flow first through the strip to form the immunocomplexes on the test and control lines while holding the flow of CL substrate. Then, the CL substrate flows sequentially through the immunocomplexes on the test and control lines to produce a CL signal. Thus, the implementation of sequential dual flows over the NC membrane is very desirable for the realization of the CL-based LFA strip. However, it has been difficult to develop an LFA strip achieving dual flows with only a one-step sample loading method which is critical for point-of-care applications, and other LFA strips and methods have not been able to develop control of dual flows in sequence over the LFA strips.

Therefore, there is a need for improved, highly sensitive rapid diagnostic tests.

### SUMMARY

Accordingly, provided herein is a strip-based RDT platform for dried or lyophilized chemiluminescence-based immunoassays, which employs sequential flow introduction. Advantageously, in embodiments, the disclosed assays comprise additional structural features that stabilize fluid flow.

According to one or more embodiments, disclosed herein is a lateral flow assay for detecting a target in a liquid sample. The lateral flow assay may comprise a first flow path and a second flow path for the liquid sample that merge at or before a membrane that generates a signal of immunoassay. The first flow path may comprise a chemiluminescent substrate pad comprising a dried or lyophilized chemiluminescent substrate, wherein the dried or lyophilized chemiluminescent substrate forms a reconstituted chemiluminescent substrate when contacted with the liquid sample, a delay pad structured and arranged to have a reduced flow velocity, and the membrane that generates the signal of immunoassay, comprising immobilized capture antibodies. The second flow path may comprise a conjugate pad comprising a dried or lyophilized detection antibody conjugated with an enzyme, wherein the dried or lyophilized detection antibody conjugated with an enzyme forms a reconstituted detection antibody conjugated with an enzyme when contacted with the liquid sample, and the membrane that generates a signal of immunoassay.

According to one or more embodiments, further disclosed herein is a lateral flow assay for detecting a target in a liquid sample. The lateral flow assay may comprise a first strip defining a first flow path. The first strip may comprise a first sample pad, a chemiluminescent substrate pad adjacent to and in fluid communication with the first sample pad, the chemiluminescent substrate pad comprising a dried or lyophilized chemiluminescent substrate, wherein the dried or lyophilized chemiluminescent substrate forms a reconstituted chemiluminescent substrate when contacted with the liquid sample, a delay pad adjacent to and in fluid communication with the chemiluminescent substrate pad, the delay pad is structured and arranged to have a reduced flow velocity, and a membrane adjacent to and in fluid communication with the delay pad comprising immobilized capture antibodies. The lateral flow assay may comprise a second strip partially overlapping the first strip and defining a second flow path. The second strip may comprise a second sample pad at least partially overlapping and in fluid communication with the first sample pad, and a conjugate pad adjacent to and in fluid communication with the second sample pad, the conjugate pad having an end extending to partially overlap and be in fluid communication with the membrane that generates a signal of immunoassay of the first strip at which point the first flow path and the second flow path merge, and the conjugate pad having a dried or lyophilized detection antibody conjugated with an enzyme, wherein the dried or lyophilized detection antibody conjugated with an enzyme forms a reconstituted detection antibody conjugated with an enzyme when contacted with the liquid sample. The first sample pad and the second sample pad may be structured and arranged to receive a liquid sample through absorption, and the liquid sample may separately flow through the first flow path and the second flow path. The lateral flow assay may be structured and arranged wherein the liquid sample flowing through the second flow path flows through the membrane that generates a signal of immunoassay for generating an immunoassay signal before the liquid sample flowing through the first flow path.

According to one or more embodiments, also disclosed herein is a lateral flow assay cartridge. The lateral flow assay cartridge may comprise a lateral flow assay for detecting a target in a liquid sample, according to one or more embodiments herein. The lateral flow assay may further comprise one or more overlaid regions wherein adjacent pads are at least partially overlaid, and a housing may enclose the lateral flow assay. The housing may further comprise one or more extended structures aligned with the one or more overlaid regions of the lateral flow assay.

According to one or more embodiments, also disclosed herein is a method for detecting a target in a liquid sample. The method may use the lateral flow assay for detecting a target in a liquid sample, according to one or more embodiments herein. The method may further comprise loading a liquid sample onto the first sample pad and the second sample pad, allowing the liquid sample to flow through the first flow path and the second flow path, and identifying any immunoassay signal.

Additional features and advantages of the articles described herein will be set forth in the detailed description which follows, and in part will be readily apparent to those skilled in the art from that description or recognized by practicing the embodiments described herein, including the detailed description which follows, the claims, as well as the appended drawings.

### BRIEF DESCRIPTION OF THE DRAWINGS

It is to be understood that both the foregoing general description and the following detailed description describe various embodiments and are intended to provide an overview or framework for understanding the nature and character of the claimed subject matter. The accompanying drawings are included to provide a further understanding of the various embodiments, and are incorporated into and constitute a part of this specification. The drawings illustrate the various embodiments described herein, and together with the description serve to explain the principles and operations of the claimed subject matter.

FIG. 1A is a flow chart schematically illustrating the dual flow concept of one or more of the lateral flow assays of the present disclosure;

FIG. 1B schematically illustrates the dual flow concept of one or more of the lateral flow assays of the present disclosure;

FIG. 2A is an exploded view showing the various layers of one or more embodiments of the lateral flow assay of the present disclosure;

FIG. 2B is a perspective view of one or more embodiments of the lateral flow assay of the present disclosure;

FIG. 2C is a perspective view of the dual flow paths of one or more embodiments of the lateral flow assay of the present disclosure;

FIG. 2D (a) schematically illustrates the dual-flow concept, (b) is a cross-sectional view showing the dual-flow paths through the lateral flow assay according to one or more embodiments of the lateral flow assay of the present disclosure, and (c) shows a perspective view showing the dual-flow paths through the lateral flow assay according to one or more embodiments of the lateral flow assay of the present disclosure;

FIG. 3 is a bar graph showing an estimation of the delay time of different membrane parameters of one or more embodiments of the delay pad of the present disclosure;

FIG. 4 is a flow chart showing a lyophilization procedure for reagent pad preparation according to one or more embodiments of the present disclosure;

FIG. 5 is a bar graph showing the delay time to reach the membrane for generating a signal of immunoassay of one or more embodiments of the lateral flow assay and cartridge as disclosed herein;

FIG. 6A is a compatibility validation of the chemiluminescent substrate with lyophilization process, showing UV-vis spectra of the CL substrate mixture before and after lyophilization, according to one or more embodiments of the present disclosure;

FIG. 6B is a compatibility validation of the chemiluminescent substrate with lyophilization process, showing the CL signal response to streptavidin-HRP surface before and after lyophilization in microchannel-based ELISA plate;

FIG. 7 is a cross-sectional photograph of a cartridge with lateral flow assay with enlarged views of the sections marked “A” and “B”, according to one or more embodiments of the present disclosure;

FIG. 8 is a cross-sectional view, top view, and bottom view of the cartridge with lateral flow assay, according to one or more embodiments of the present disclosure;

FIG. 9 is a series of sequential photographs showing color changes that appeared on the detection window of the cartridge over time, according to one or more embodiments of the present disclosure; and

FIG. 10 is a graph showing chemiluminescence-based cTnI assay results on a 96-well plate, a commercialized LFA that is not dual-flow, and a lateral flow assay according to one or more embodiments of the present disclosure.

Reference will now be made in greater detail to various embodiments of the present disclosure, some embodiments of which are illustrated in the accompanying drawings. Whenever possible, the same reference numerals will be used throughout the drawings to refer to the same or similar parts.

### DETAILED DESCRIPTION

Described herein are lateral flow assays 10 for detecting a target antigen in a liquid sample. In embodiments described herein, the lateral flow assay 10 includes multiple flow paths where at least one flow path includes a chemiluminescent substrate pad and membrane for generating a signal of immunoassay. The present disclosure is also directed to cartridges 20 that include the lateral flow assay 10, and methods of detecting a target antigen using the lateral flow assay 10. These embodiments are described in detail herein.

As discussed above, a disadvantage of prior lateral flow assays is a lower sensitivity attributable to poor response in low concentrations, which makes the prior lateral flow assays unattractive for diagnosing low-abundant biomarkers. Further, chemiluminescent assays that have a lower limit-of-detection are difficult to include in a lateral flow assay because of the lack of methods for storing liquid CL substrates and realizing the desired sequential dual flows on the LFA strip at the same time, as well as achieving dual flows with only a one-step sample loading method advantageous for point-of-care applications.

The present disclosure aims to solve this problem by including structural features in the lateral flow assay that achieve controllable sequential dual-flow of the liquid sample.

In the present disclosure, dried or lyophilized chemiluminescent substrate and dried or lyophilized detection antibody conjugated with an enzyme are reconstituted via contact with a liquid sample and travel along different flow paths, with the reconstituted chemiluminescent substrate flow path being delayed by a delay pad included in the flow path. This delay allows the antibody conjugated with an enzyme to contact immobilized capture antibodies on the membrane before contacting the chemiluminescent substrate, so that the chemiluminescent substrate can react with the conjugated enzyme upon reaching immobilized capture antibodies and antibody conjugated with the enzyme to generate the immunoassay signal. The lateral flow assay allows for highly sensitive detection or the target antigen in a simple one-step assay.

Various non-limiting embodiments of the present disclosure will now be described to provide an overall understanding of the principles of the structure, function, and use of the apparatuses, systems, methods, and processes disclosed herein. One or more examples of these non-limiting embodiments are illustrated in the accompanying drawings. Those of ordinary skill in the art will understand that systems and methods specifically described herein and illustrated in the accompanying drawings are non-limiting embodiments. The features illustrated or described in connection with one non-limiting embodiment may be combined with the features of other non-limiting embodiments. Such modifications and variations are intended to be included within the scope of the present disclosure.

Described herein is a lateral flow assay 10 for detecting a target antigen in a liquid sample. The lateral flow assay may comprise multiple flow paths for the liquid sample that merge at or before a membrane that generates a signal of immunoassay 300. For example, the lateral flow assay 10 may include a first flow path 100 and a second flow path 200 for the liquid sample that merge at or before a membrane that generates a signal of immunoassay 300. In some embodiments, the first flow path 100 and the second flow path 200 are defined by separate strips of materials (e.g., membranes), such as a first strip 105 and a second strip 205 that may at least partially overlap and that merge at or before the membrane that generates a signal of immunoassay 300.

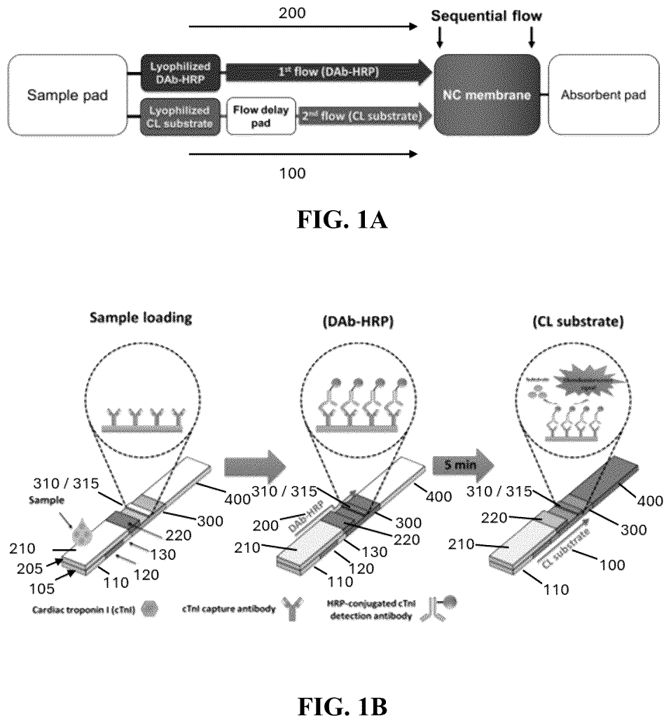

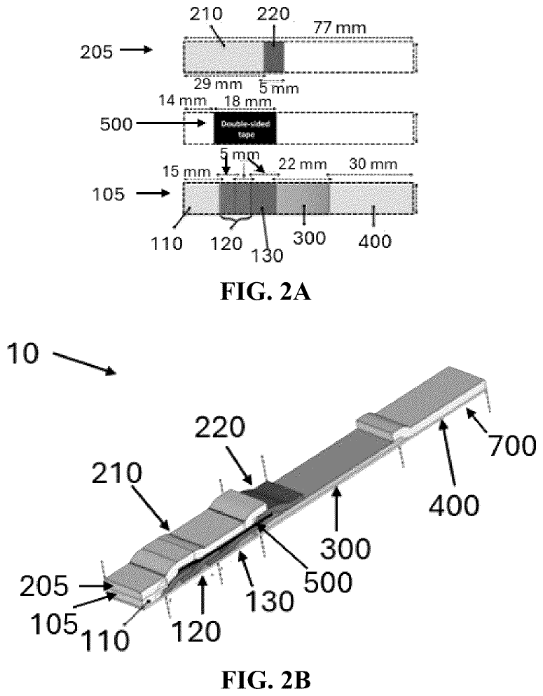

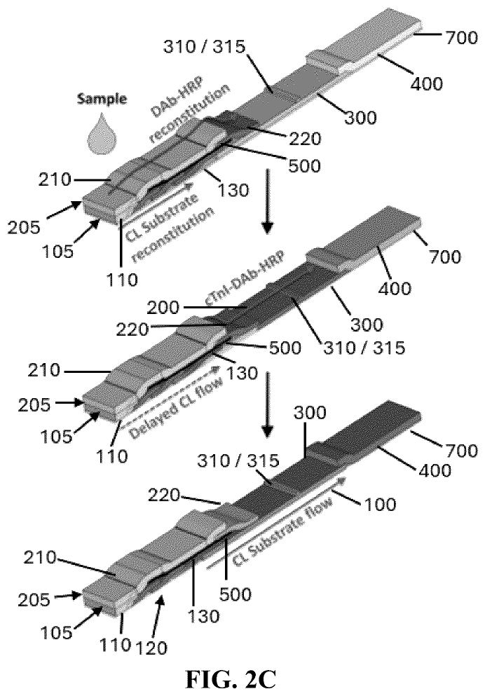

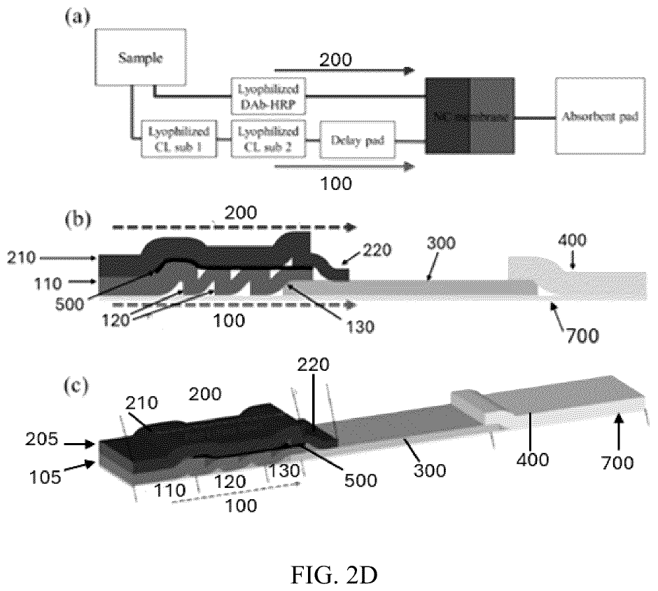

In some embodiments described herein, as shown in FIGS. 1B and 2A-2D, the first strip 105 defining the first flow path 100 may comprise a chemiluminescent substrate pad 120, a delay pad 130, and the membrane that generates a signal of immunoassay 300. The first flow path may further comprise a sample pad, such as a first sample pad 110, and a wicking pad 400 structured and arranged to absorb excess liquid sample.

In some embodiments described herein, as shown in FIGS. 1B and 2A-2D, the second strip 205 defining the second flow path 200 may comprise a conjugate pad 220 comprising a dried or lyophilized detection antibody conjugated with an enzyme and merges at or before the membrane that generates a signal of immunoassay 300. The second flow path may further comprise a sample pad, such as a second sample pad 210, and may further travel to the wicking pad 400 after passing through the membrane that generates a signal of immunoassay 300.

In some embodiments, the lateral flow assay 10 includes at least two flow paths in which the liquid sample passes through simultaneously, but which arrive at the membrane 300 sequentially with the liquid sample flowing through the second flow path 200 arriving first and the liquid sample flowing through the first flow path 100 and arriving subsequently. The dual-flow nature of the lateral flow assay 10 allows for a chemiluminescence-based immunoassay with a single-time sample loading using dried or lyophilized chemiluminescent substrate. An exemplary concept of operation of the lateral flow assay 10 including the dual flow paths and the sequential flows for a target antigen is illustrated in FIGS. 1A, 1B, 2C, and 2D. As shown in FIGS. 1B and 2A-2D, the lateral flow assay 10 strip includes a first flow path 100 defined by a first strip 105 including a first sample pad 110, a chemiluminescent substrate pad 120 containing a dried or lyophilized chemiluminescent substrate, the membrane that generates a signal of immunoassay 300 that includes immobilized capture antibodies (CAb) at a defined area, for example a test and control line, of the membrane 300, and a wicking pad 400, and a second flow path 200 defined by a second strip 205 over the first strip 105 including a second sample pad 210 and a conjugate pad 220 that is in fluid communication with the membrane 300 at which point the first flow path 100 and the second flow path 200 merge. In some embodiments, the second flow path 200 may merge with the first flow path 100 before the membrane 300. The conjugate pad 220 contains a detection antibody (DAb) for the target antigen conjugated with an enzyme, such as horseradish peroxidase (HRP). In order to realize the sequential introduction of the DAb-HRP first and the chemiluminescent substrate later, the flow timing and delay of chemiluminescent substrate is structured and arranged to be controlled using a delay pad 130 in the first strip 105 defining the first flow 100 path as shown in FIGS. 2A and 2B. Accordingly, the dried or lyophilized DAb-HRP and chemiluminescent substrates in each layer (e.g., upper second strip 205 and lower first strip 105) were concurrently reconstituted by the sample liquid but sequentially introduced to the membrane that generates a signal of immunoassay 300, as depicted in FIGS. 1A and 2C. Thus, the reconstituted DAb-HRP flowed first through the second flow path 200 of the second strip 205 of the lateral flow assay 10 test strip to form the immunocomplexes of CAb-target antigen-DAb-HRP. After a time period, such as 5 minutes from the sample loading, the reconstituted chemiluminescent substrate in the sample liquid in the first flow path 100 of the first strip 105 reaches the test line 315 and the control line 325 on the membrane 300 and produces optical chemiluminescent signals via reaction with HRP. The optical chemiluminescent signal may be measured using a microplate reader, a luminometer or other suitable device.

As discussed above, the first flow path 100 comprises a chemiluminescent substrate pad 120. In some embodiments, the chemiluminescent substrate pad 120 comprises a dried or lyophilized chemiluminescent substrate. The dried or lyophilized chemiluminescent substrate includes a chemiluminescent reagent that is a compound capable of chemical reaction to form products along with simultaneous emission of light. The chemiluminescent reagent may comprise luminol, a luminol-based compound, acridan, an acridan-based compound, as 1,2-dioxetane-based compound, or the like, and peroxide, peroxide generator, hydrogen peroxide, urea peroxide, sodium perborate, sodium percarbonate, or the like. It should be appreciated that many chemiluminescent reagents are known in the field and suitable for use in the presently disclosed devices.

The first flow path 100 further comprises a delay pad 130. The delay pad 130 may be structured and arranged to have a reduced flow velocity. The reduced flow velocity may be relative to the other flow components of the lateral flow assay 10. For example, as discussed above, the delay pad 130 may reduce the flow velocity of the liquid sample through the first flow path 100 relative to the flow velocity of the liquid sample through the second flow path 200 wherein the liquid sample flowing through the second flow path 200 flows through the membrane for generating an immunoassay signal 300 before the liquid sample flowing through the first flow path 100. In one or more embodiments, the time for the liquid sample to flow through the first flow path 100 may be at least 2 minutes longer than the time for the liquid sample to flow through the second flow path 200, or at least 5 minutes longer, or at least 8 minutes longer, or at least 10 minutes longer. In one or more embodiments, the time for the liquid sample to flow through the first flow path 100 may be 2 to 20 minutes longer than the time for the liquid sample to flow through the second flow path 200, or 2 to 15 minutes longer, or 2 to 10 minutes longer, or 5 to 20 minutes longer, or 5 to 15 minutes longer, or 5 to 10 minutes longer, or 8 to 20 minutes longer, or 8 to 15 minutes longer, or 8 to 10 minutes longer.

The delay pad 130 may be structured and arranged to have a reduced flow velocity through selection of its composition (e.g., hydrophobicity or hydrophilicity) and pore size. In some embodiments, the delay time from the delay pad 130 may be estimated based on the delay pad 130 characteristics using the Lucas-Washburn equation that describes penetration length of a membrane by a liquid in a given time, as expressed in Equation 1 below:

$$L = \sqrt{\frac{γ rt cos θ}{2 η}} \qquad ( 1 )$$

where L is the penetration length of the membrane, γ is the surface tension of the liquid, r is the pore radius of the membrane, t is the time spent for penetration, θ is the contact angle of a liquid to the membrane, and η is the dynamic viscosity of the liquid. According to the equation, the penetration length through a membrane is governed by several parameters, such as the membrane and liquid properties mentioned above. Accordingly, a membrane with a lower contact angle facilitates flow penetration through the membrane, while a contact angle greater than 90 degrees does not allow the flow to penetrate. Thus, the penetration time can be controlled by modifying the properties of the delay pad 130 in terms of surface hydrophilicity, pore size, length, etc. Accordingly, the delay pad 130 appropriate for the lateral flow assay 10 may be selected based upon the length, pore size, and hydrophilicity of the delay pad 130, and may further depend upon the source of the sample.

In some embodiments, the delay pad 130 may have a pore size of from 0.05 μm to 5 μm, or from 0.05 μm to 1 μm, or from 0.05 μm to 0.5 μm, or from 0.05 μm to 0.1 μm, or from 0.1 μm to 5 μm, or from 0.1 μm to 1 μm, or from 0.1 μm to 0.5 μm, or from 0.5 μm to 5 μm, or from 0.5 μm to 1 μm.

In some embodiments, the delay pad 130 may have a water contact angle of at least 20 degrees, at least 35 degrees, at least 50 degrees, at least 65 degrees, or at least 80 degrees. In some embodiments, the delay pad 130 may have a water contact angle of no more than 89 degrees, no more than 80 degrees, no more than 65 degrees, no more than 50 degrees, no more than 35 degrees, or no more than 20 degrees. In some embodiments, the delay pad 130 may have a water contact angle of 20 to 89 degrees, 20 to 80 degrees, 20 to 65 degrees, 20 to 50 degrees, 20 to 35 degrees, 35 to 89 degrees, 35 to 80 degrees, 35 to 65 degrees, 35 to 50 degrees, 50 to 89 degrees, 50 to 80 degrees, 50 to 65 degrees, 65 to 89 degrees, 65 to 80 degrees, or 80 to 89 degrees. In some embodiments, the delay pad 130 may be a semi-hydrophilic material having a water contact angle of 35 to 65 degrees. As used herein, the term “semi-hydrophilic” refers to materials that exhibit an intermediate water affinity where they exhibit partial but not complete attraction to water such that they tend to be wet by water but less than a hydrophilic surface.

In some embodiments, the delay pad 130 may comprise or be selected from the group consisting of aluminum oxide, cellulose acetate, cellulose ester, glass fiber, nitrocellulose, Nylon, polyacrylonitrile, polycarbonate, polyethersulfone, polyethylene, polyethylene terephthalate, polypropylene, polytetrafluoroethylene, and polyvinylidene difluoride (PVDF).

In one or more embodiments, the delay pad 130 may comprise a semi-hydrophilic membrane having a water contact angle of 35 to 65 degrees, or about 50 degrees, such as PVDF, and a pore size between 0.1 μm and 1 μm may be used for the delay pad.

In embodiments, as shown in FIGS. 1B and 2C, the first flow path 100, and second flow path 200 once merged, further comprise a membrane for generating an immunoassay signal 300. The membrane for generating an immunoassay signal 300 comprises a zone of indication 310 having immobilized capture antibodies specific to the target antigen such that the capture antibodies may bind to the antigen-DAb-enzyme (e.g., HRP) complex. The zone of indication 310 may be any suitable form, such as a test indicator line 315 or any other suitable shape. The membrane for generating an immunoassay signal 300 further comprises a zone of control indication 320 (not shown) that produces a control signal. The zone of control indication 320 may include immobilized secondary antibodies that may bind with DAb-enzyme that has not complexed with the antigen. The zone of control indication 320 may be any suitable form, such as an indicator line 325 (not shown) or any other suitable shape.

The membrane for generating an immunoassay signal 300 may be formed from synthetic or naturally occurring materials, such as polysaccharides (e.g., cellulose materials such as paper and cellulose derivatives, such as cellulose acetate and nitrocellulose); polyether sulfone; polyethylene; nylon; polyvinylidene fluoride (PVDF); polyester; polypropylene; silica; inorganic materials, such as deactivated alumina, diatomaceous earth, magnesium sulfate, or other inorganic finely divided material uniformly dispersed in a porous polymer matrix, with polymers such as vinyl chloride, vinyl chloride-propylene copolymer, and vinyl chloride-vinyl acetate copolymer; cloth, both naturally occurring (e.g., cotton) and synthetic (e.g., nylon or rayon); porous gels, such as silica gel, agarose, dextran, and gelatin; polymeric films, such as polyacrylamide; and so forth. In one particular embodiment, the membrane 300 is formed from nitrocellulose. It should be understood that the term “nitrocellulose” refers to nitric acid esters of cellulose, which may be nitrocellulose alone, or a mixed ester of nitric acid and other acids, such as aliphatic carboxylic acids having from 1 to 7 carbon atoms.

The membrane for generating an immunoassay signal 300 may be any suitable size, such as those described for the exemplary lateral flow assays 10 herein. The thickness of the membrane for generating an immunoassay signal 300 may be generally small enough to allow for chemiluminescent detection. For example, the membrane 300 may have a thickness less than 500 micrometers, in some embodiments less than 250 micrometers, or in some embodiments, less than 150 micrometers.

In some embodiments described herein, as shown in FIGS. 1B and 2A-2D, the second flow path 200 may comprise a conjugate pad 220 comprising a dried or lyophilized detection antibody conjugated with an enzyme. The detection antibody (DAb) is not limited and may be selected based upon the target antigen. For example, as used in the Examples section of the present disclosure, the detection antibody may be a detection antibody for cardiac troponin I (cTnI); however, the present disclosure is not limited thereto and the detection antibody may be selected based upon its binding capacity for various target antigens. The lateral flow assay 10 of the present disclosure may be particularly suitable for antigens where case of use and/or an enhanced limit of detection is desired.

In some embodiments, the detection antibody is conjugated with an enzyme. The enzyme is not limited so long as it is capable of reacting, catalyzing, or otherwise interacting with the chemiluminescent substrate to generate a chemiluminescent signal. Non-limiting examples include horseradish peroxidase (HRP), alkaline phosphatase (AP), soybean peroxidase (SBP), or the like. For example, in the case of a chemiluminescent substrate comprising luminol as a chemiluminescent reagent and peroxide as a co-reagent, horseradish peroxidase may catalyze the reaction of luminol and peroxide wherein the reaction results in the emission of light.

Exemplary methods of preparing the conjugate pad 220 having dried or lyophilized detection antibody conjugated with an enzyme are provided in the Examples section below.

In embodiments, as discussed above, the lateral flow assay 10 further comprises at least one sample pad. The sample pad may be positioned at one end as the terminal portion of the strip of the lateral flow assay 10 and is structured and arranged to receive a liquid sample. The sample pad may comprise a single pad that is in fluid communication with the first flow path 100 and second flow path 200, or, alternatively, may be multiple pads that at least partially overlap. The liquid sample may be absorbed through first sample pad and second sample pad (or a single sample pad) which are in fluid communication with the first flow path 100 and the second flow path 200, respectively. The sample pad is not limited and could include any suitable porous material. Non-limiting examples of suitable sample pads include nitrocellulose, cellulose, porous polyethylene pads, glass fiber filter paper, and the like.

In embodiments, as shown in FIGS. 1B and 2A-2D, and as discussed above, the lateral flow assay 10 may further comprise a wicking pad 400. The wicking pad 400 is positioned at an end 152 opposite the sample pad as the terminal portion of the strip of the lateral flow assay 10. In some embodiments, the wicking pad 400 may be adjacent to the membrane for generating an immunoassay signal 300, and the wicking pad 400 may be structured and arranged to absorb any excess liquid sample that travels the length of the membrane 300. The wicking pad 400 is not limited and may include any suitable porous material. Non-limiting examples of materials suitable for the wicking pad 400 include nitrocellulose, cellulosic materials, porous polyethylene pads, glass fiber filter paper, and the like.

In one or more embodiments, still referring to FIGS. 2A-2D, the lateral flow assay 10 may further comprise a separation film 500 positioned between the first strip 105 defining the first flow path 100 and the second strip 205 defining the second flow path 200. The separation film 500 may be positioned to separate the chemiluminescent substrate pad 120 and the delay pad 130 from fluidly communicating with the second strip 205. In doing so, the separation film 500 prevents the liquid sample traveling through the first flow path 100, including with the reconstituted chemiluminescent substrate, from entering the second flow path 200 and ensures that the reconstituted chemiluminescent substrate must flow through the delay pad 130 to arrive at the zone of indication 310 after the detection antibody conjugated with an enzyme such that the reconstituted chemiluminescent substrate may interact with the enzyme to generate the chemiluminescent signal at the zone of indication 310. Accordingly, in some embodiments, the separation film 500 is present at least between the length of the chemiluminescent substrate pad 120 and the delay pad 130 and the second flow path 200. In some embodiments, the separation film may also extend beyond the chemiluminescent substrate pad 120 and delay pad 130 of the first strip 105 of the first flow path 100 over a portion of the membrane for generating an immunoassay signal 300; however, the separation film 500 does not extend the entire length of the conjugate pad 220 of the second strip 205 of the second flow path 200 as at least a portion of the conjugate pad 220 must extend further than the separation film 500 such that the conjugate pad 220 may fluidly communication at or before the membrane for generating an immunoassay signal 300 to merge the first flow path 100 and the second flow path 200. As shown in FIGS. 2A-2D, the separation film 500 may extend over the length of the chemiluminescent substrate pads 120, delay pad 130, and a portion of the membrane for generating an immunoassay signal 300, and the conjugate pad 220 may extend past the separation film 500 to contact the membrane for generating an immunoassay signal 300. The separation film 500 generally does not separate the first sample pad 110 and the second sample pad 210; however, partial separation may occur in some embodiments. In some embodiments, the separation pad 500 extends from and end of the first sample pad 110 over the chemiluminescent substrate pad 120 and the delay pad 130 to the membrane 300, and may partially extend over the membrane 300, as shown in FIG. 2A. The separation film 500 may be any suitable material. In some embodiments, for example, the separation film 500 may be a double sided tape.

In one or more embodiments, as shown in FIGS. 2B, 2C, and 2D, the lateral flow assay 10 may further comprise a support 700. In some embodiments, the support 700 may be affixed to the first strip 105 defining the first flow path 100. The support 700 may be any suitable backing material. For example, in some embodiments, the support 700 may be a polymeric backing material having an adhesive on one side for adhering to the first strip 105 of the lateral flow assay 10 to the support 700.

In one or more embodiments, as shown in FIGS. 1B and 2A-2D, a first strip 105 defining a first flow path 100 may comprise a first sample pad 110, a chemiluminescent substrate pad 120 adjacent to and in fluid communication with the first sample pad 110, the chemiluminescent substrate pad 120 comprising a dried or lyophilized chemiluminescent substrate, wherein the dried or lyophilized chemiluminescent substrate forms a reconstituted chemiluminescent substrate when contacted with the liquid sample, a delay pad 130 adjacent to and in fluid communication with the chemiluminescent substrate pad 120, the delay pad 130 may be structured and arranged to have a reduced flow velocity, a membrane for generating an immunoassay signal 300 adjacent to and in fluid communication with the delay pad 130, the membrane 300 comprising immobilized capture antibodies, and a wicking pad 400 adjacent to and in fluid communication with the membrane for generating an immunoassay signal 300.

In one or more embodiments, as shown in FIGS. 1B and 2A-2D, a second strip 205 defining a second flow path 200 may comprise a second sample pad 210 at least partially overlapping and in fluid communication with the first sample pad 110, and a conjugate pad 220 adjacent to and in fluid communication with the second sample pad 210, the conjugate pad 220 having an end extending to partially overlap and be in fluid communication with the membrane 300 of the first strip 105 at which point the first flow path 100 and the second flow path 200 merge, and the conjugate pad 220 having the dried or lyophilized detection antibody conjugated with an enzyme, wherein the dried or lyophilized detection antibody conjugated with an enzyme forms a reconstituted detection antibody conjugated with an enzyme when contacted with the liquid sample.

In one or more embodiments, the first sample pad 110 and the second sample pad 210 are structured and arranged to receive a liquid sample through absorption, and the liquid sample separately flows from the first sample pad 110 and the second sample pad 210 through the first flow path 100 and the second flow path 200, respectively.

In one or more embodiments, the lateral flow assay 10 may be structured and arranged wherein the liquid sample flowing through the second flow path 200 flows through the membrane for generating an immunoassay signal 300 before the liquid sample flowing through the first flow path 100 flow through the membrane 300.

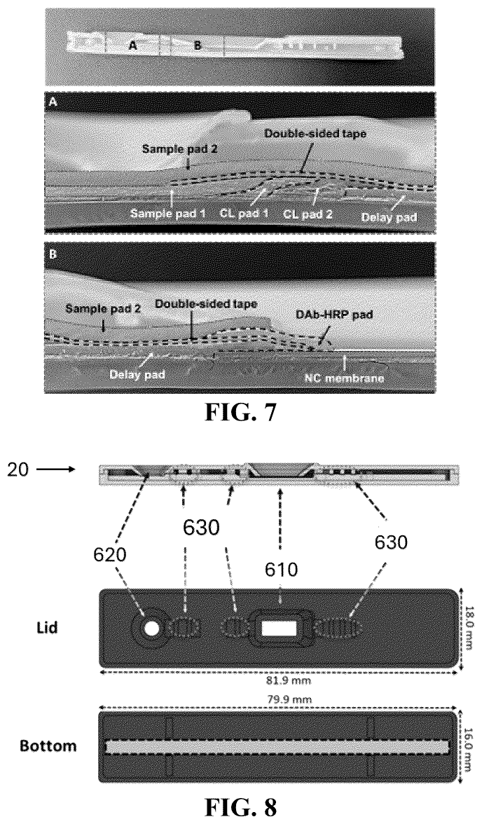

In one or more embodiments, as shown in FIGS. 2B, 2C, 2D, and 7, the components of the first strip 105 and the second strip 205 may be overlaid with each other to facilitate flow between the components to form one or more overlaid regions. For example, in the first strip 105, the adjacent ends of first sample pad 110 and the chemiluminescent substrate pad 120 may be at least partially overlaid, and the adjacent ends of the chemiluminescent substrate pad 120 and the delay pad 130 may be at least partially overlaid, the adjacent ends of the delay pad 130 and the membrane 300 may be at least partially overlaid, and the adjacent ends of the membrane 300 and the wicking pad 400, if present, may be at least partially overlaid. Likewise, in the second strip 205, the adjacent ends of the second sample pad 210 and the conjugate pad 220 may be at least partially overlaid, and the adjacent ends of the conjugate pad 220 and the membrane 300 may be at least partially overlaid. The amount of overlay in the overlaid regions may be consistent between all components or may vary. For example, the amount of overlay may be at least 0.1 mm, at least 0.5 mm, at least 1 mm, at least 2 mm, or more, such as from 0.1 mm to 5 mm, from 0.1 mm to 2 mm, from 0.1 mm to 1 mm, from 0.1 mm to 0.5 mm, from 0.5 mm to 5 mm, from 0.5 mm to 2 mm, from 0.5 mm to 1 mm, from 1 mm to 5 mm, from 1 mm to 2 mm, or from 2 mm to 5 mm. In addition, in some embodiments, the individual components may include more than 1 pad that are likewise overlaid. For example, as shown in FIG. 2A, the chemiluminescent substrate pad 120 may comprise two or more pads that are overlaid with each other. In one or more embodiments, as shown in FIGS. 7 and 8, the housing 600 of cartridge 20 may comprise one or more extended structures 630 that are aligned with one or more of the overlaid regions and serve to pinch and apply pressure to the overlaid regions of the lateral flow assay 10 to ensure contact between adjacent components.

In some embodiments, still referring to FIG. 8, the cartridge 20 further comprises a sample loading port 610 positioned over the sample pad, such as the first sample pad 110 and the second sample pad 210, for receiving the liquid sample. The sample loading port 610 is not limited so long as it allows enough access to the sample pad to load the liquid sample.

In some embodiments, still referring to FIG. 8, the cartridge 20 further comprises a detection window 620 positioned over the membrane that generates a signal of immunoassay 300. The detection window 620 allows for viewing and analysis of the chemiluminescent signal generated during use of the lateral flow assay 10 in the cartridge 20. The detection window 620 is not limited so long as it allows for viewing of the zone of indication 310, such as a test line 315, and optionally the zone of control indication 320, such as a control line 325. In some embodiments, the detection window 620 may include two or more windows for observing multiple zones of indication 310 and/or zones of control indication 320.

Also disclosed is a method for detecting a target in a liquid sample using the lateral flow assay one or more embodiments of the present disclosure. The method may comprise loading a liquid sample to the sample pad, such as the first sample pad and the second sample pad, allowing the liquid sample to flow through the first flow path and the second flow path, and identifying any immunoassay signal. In one or more embodiments, the liquid sample may be loaded in a single step without requiring additional separate loadings of the liquid sample, the chemiluminescent substrate, and/or the detection antibody conjugated with an enzyme.

The terms “free” and “substantially free,” when used to describe the concentration and/or absence of a particular constituent component means that the constituent component is not intentionally added.

Ranges can be expressed herein as from “less than or equal to” one particular value, and/or to “less than or equal to” another particular value. When such a range is expressed, another embodiment includes from the one particular value and/or to the other particular value. Similarly, when values are expressed as approximations, by use of the antecedent “less than or equal to,” it will be understood that the particular value forms another embodiment. It will be further understood that the endpoints of each of the ranges are significant both in relation to the other endpoint, and independently of the other endpoint. Any ranges used herein include all ranges and subranges and any values there between unless explicitly stated otherwise.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

Directional terms as used herein—for example up, down, right, left, front, back, top, bottom—are made only with reference to the figures as drawn and are not intended to imply absolute orientation.

Unless otherwise expressly stated, it is in no way intended that any method set forth herein be construed as requiring that its steps be performed in a specific order, nor that with any apparatus specific orientations be required. Accordingly, where a method claim does not actually recite an order to be followed by its steps, or that any apparatus claim does not actually recite an order or orientation to individual components, or it is not otherwise specifically stated in the claims or description that the steps are to be limited to a specific order, or that a specific order or orientation to components of an apparatus is not recited, it is in no way intended that an order or orientation be inferred, in any respect. This holds for any possible non-express basis for interpretation, including: matters of logic with respect to arrangement of steps, operational flow, order of components, or orientation of components; plain meaning derived from grammatical organization or punctuation, and; the number or type of embodiments described in the specification.

As used herein, the singular forms “a,” “an” and “the” include plural referents unless the context clearly dictates otherwise. Thus, for example, reference to “a” component includes aspects having two or more such components, unless the context clearly indicates otherwise.

Reference throughout the specification to “various embodiments,” “some embodiments,” “one embodiment,” “some example embodiments,” “one example embodiment,” or “an embodiment” means that a particular feature, structure, or characteristic described in connection with any embodiment is included in at least one embodiment. Thus, appearances of the phrases “in various embodiments,” “in some embodiments,” “in one embodiment,” “some example embodiments,” “one example embodiment,” or “in an embodiment” in places throughout the specification are not necessarily all referring to the same embodiment. Furthermore, the particular features, structures or characteristics may be combined in any suitable manner in one or more embodiments.

In various embodiments disclosed herein, a single component can be replaced by multiple components and multiple components can be replaced by a single component to perform a given function or functions. Except where such substitution would not be operative, such substitution is within the intended scope of the embodiments.

The examples discussed herein are examples only and are provided to assist in the explanation of the apparatuses, devices, systems and methods described herein. None of the features or components shown in the drawings or discussed below should be taken as mandatory for any specific implementation of any of these the apparatuses, devices, systems or methods unless specifically designated as mandatory. For case of reading and clarity, certain components, modules, or methods may be described solely in connection with a specific figure. Any failure to specifically describe a combination or sub-combination of components should not be understood as an indication that any combination or sub-combination is not possible. Also, for any methods described, regardless of whether the method is described in conjunction with a flow diagram, it should be understood that unless otherwise specified or required by context, any explicit or implicit ordering of steps performed in the execution of a method does not imply that those steps must be performed in the order presented but instead may be performed in a different order or in parallel.

Having shown and described various versions in the present disclosure, further adaptations of the methods and systems described herein may be accomplished by appropriate modifications by one of ordinary skill in the art without departing from the scope of the present disclosure. Several of such potential modifications have been mentioned, and others will be apparent to those skilled in the art. For instance, the examples, versions, geometrics, materials, dimensions, ratios, steps, and the like discussed above are illustrative and are not required. Accordingly, the scope of the present disclosure should be considered in terms of the following claims and is understood not to be limited to the details of structure and operation shown and described in the specification and drawings.

### EXAMPLES

The various embodiments of systems and processes of the present disclosure will be further clarified by the following examples. The examples are illustrative in nature, and should not be understood to limit the subject matter of the present disclosure.

### Example 1: Membrane Characteristics and Selection for Sequential Dual Flows

Lateral flow assays (LFA) were prepared according to the structure shown in FIG. 2A. The first strip for the first flow path included a sample pad and a DAb-HRP pad. The second strip for the second flow path included another sample pad, chemiluminescent (CL) substrate pads, and a delay pad. The upper and lower strips were physically separated into the first flow path and the second flow path using a double-sided adhesive tape (ARCare 94119, Adhesive Research, USA) to ensure the separation of flows through each flow path. Thus, the flows of the first flow path and the second flow path reached and flow through the nitrocellulose (NC) membrane at different times. The detailed flow sequence in an isometric view is illustrated in FIGS. 1B and 2C.

In the flow test of the lateral flow assay strips, the flowing liquid sample simultaneously reconstituted both the lyophilized DAb-HRP and the dried CL substrate in each path. And then, the reconstituted DAb-HRP in the first strip for the first flow path flowed over the NC membrane first and was captured by the immobilized capture antibodies (CAb), producing CAb-cTnI-DAb-HRP immunocomplexes. On the other hand, the reconstituted CL substrate in the lower strip (path 2) was tentatively held and delayed by the flow delay pad and then flowed over the formed immunocomplexes to produce the optical CL signal. In other words, the cTnI-DAb-HRP flow reached the test and control lines on the NC membrane while the CL substrate in the lower strip was flowing slowly through the delay pad. After having the desired delay time, the CL substrate in the lower strip passed through the delay pad and reached the test and control lines, and the optical CL signal was produced as the product of reaction between the HRP and CL substrate.

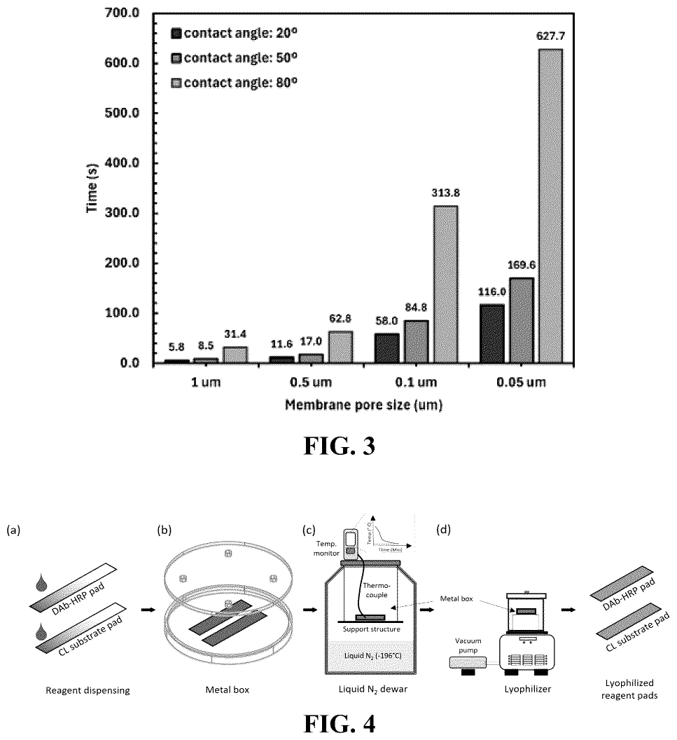

Various membrane candidates were considered for the delay pad for use with human serum as the liquid sample, including those with pore sizes of between 0.05 μm and 5 μm. The length of the delay pad was fixed at 10 mm due to the following restrictions in the strip dimension. First, the total length of the lateral flow assay (LFA) strip was fixed at 77 mm so as to fit into an LFA cartridge 20 of 80 mm in length. Second, the location of the delay pad was set to allow a 2 mm of margin from the front end of a detection window on the LFA cartridge. The third restriction came from the sample loading port, which resulted in 2 mm of margin from the end of the sample loading port. As a result, those three restrictions allowed a 12 mm length for two chemiluminescent substrate pads and a delay pad. Considering 2 mm of overlays in every interface between membranes, 5 mm was the desired minimum length of a membrane for the delay pad. In order to maximize the length of the flow delay pad, two chemiluminescent substrate pads, each 5 mm long, and a 10 mm delay pad were designed for the lower first strip. The dimensions of the strips designed with the design restrictions as shown in FIG. 2A. FIG. 3 shows the calculated delay times using Equation 1 with various combinations of membrane characteristics, including membrane pore sizes and contact angles. The liquid was assumed to be a human serum, where the surface tension was 58.7 mN/m and dynamic viscosity was 1.6 cP. FIG. 3 shows that the selection of pore sizes was more effective in achieving the desired delay time, although the semi-hydrophilic nature of the delay pad composition facilitates a longer delay. In addition, penetration time was also altered due to the tape cover on the top of the membrane of the delay pad. In view of these results, a semi-hydrophilic membrane with a pore size between 0.1 μm and 1 μm was chosen for over the delay pad in these Examples.

### Example 2: Characterization of Materials and Preparation of Dual-Flow Lateral Flow Assay Strips for Detecting Cardiac Troponin 1

### Dual-Flow Lateral Flow Assay Strip Preparation and Assembly

The dimensions of membranes for the upper and lower strips are shown in FIG. 2A, and the preparation steps of the dual-flow LFA strips with CL-based immunoassay was as follows. In the first step for the upper strip preparation, the detection antibody (4T21cc-MF4cc, Hytest, Finland) was conjugated with HRP using a Lightning-link HRP conjugation kit (ab 102890, Abcam, USA). The DAb-HRP pad (Fusion 5, Cytiva, USA) was cut to a size of 100 mm×5 mm, and 211.5 μl of 1 μg/ml DAb-HRP solution, diluted in StartingBlock (37578, ThermoFisher Scientific, USA), was dispensed over the pad. The lyophilization steps used for DAb-HRP and CL substrate pads are illustrated in FIG. 4, wherein in step (a) reagent pads were prepared by dispensing desired reagents, in step (b) the pads were put into a metal box that maintains the temperature during lyophilization step (c), step (c) illustrates the freezing step where the metal box was maintained in the liquid nitrogen dewar until the temperature reached −160° C.; and step (d) illustrates the lyophilization step wherein the metal box is transferred to the lyophilizer. After lyophilization step, the pads with lyophilized reagents were used for dual flow LFA strip assembly. The DAb-HRP pad was located in an aluminum container, and then it was frozen in liquid nitrogen dewar (35VHC, Taylor-Wharton, USA). After the temperature on the thermometer reached −160° C., the aluminum container was moved to the lyophilization chamber of a Labconco lyophilizer (Freezone 1), and then lyophilized at −54° C. and 0.01 mbar for 12 hours. For the lower strip, two CL substrate pads (Fusion 5) were cut to a size of 100 mm×5 mm using a manual cutter, then followed by dispensing 211.5 ul of CL substrates (SuperSignal ELISA Femto, ThermoFisher Scientific, USA), luminol and peroxide solution, separately. The lyophilization steps used for the CL substrate were the same as the DAb-HRP pad. Then, the pads were cut to a size of 70 mm×22 mm using a manual cutter. Capture antibodies (4T21cc-16A11cc and 4T21cc-4C2cc, Hytest, Finland) at the concentration of 1 mg/ml in mixture were dispensed on the NC membrane using a syringe pump (Dual 33, Havard apparatus, USA) and USB-controlled XYZ stage (Vision M1, Dunwell Tech Inc., USA), which resulted in 0.4 μg of capture antibody on the test line. Then, the NC membrane was dried at 37° C. in a convection oven for 2 hours. All other components, such as the double-sided adhesive tape, the delay pad (Hydrophilic PVDF, 0.45 microns, Sterlitech, USA), the absorption pad (CF4, Whatman), and the upper and lower sample pads (Fusion 5), were cut into the desired dimensions using a manual cutter. Finally, the prepared NC membrane was attached first to a backing card support (KN-2211, Kenosha Tapes, Netherlands), followed by an absorption pad, CL substrate pads, and a lower sample pad. Every pad was overlaid by 2 mm each other to facilitate the flow between pads. After the double-sided tape was attached to the strip, the DAb-HRP pad and upper sample pad were added to complete the assembly. Then, the assembled membranes were cut with a manual cutter at a 4 mm width, and each cut was used as the dual-flow LFA strip.

### Evaluation of Delay Time Using Various Membranes for Delay Pad

In order to compare and select delay pads, various materials were considered, as shown in Table 1. Artificial serum (CST Technologies Inc., USA) with blue dye solution was used as a sample, and the evaluation was performed with the lower strip layer of the dual flow LFA strips which were composed of a lower sample pad, CL substrate pads, delay pad, NC membrane, absorbent pad, and double-sided tape. After 80 μl of blue-colored artificial serum solution was loaded at the sample loading port, the time until the flow reached the detection window was measured and analyzed.

TABLE 1
List of membrane candidates for delay pad such as Polyethersulfone
(PES), Polyvinylidene difluoride (PVDF), and Polycarbonate (PC)

| | | | | Pore size | Wicking rate |
|---|---|---|---|---|---|
| Manufacturer | Product name | Material | Surface | (μm) | (s/4 cm) |
| Whatman | Fusion 5 | Proprietary | Hydrophilic | N/A | 43.9 |
| Ahlstrom | ReliaFlow 238 | Glass fibers | Hydrophilic | N/A | 150 |
| | ReliaFlow 601 | Glass fibers | Hydrophilic | N/A | 120 |
| Sterlitech | PES | PES | Hydrophilic | 0.03 | N/A |
| | Hydrophilic PVDF | PVDF | Hydrophilic | 0.45 | N/A |
| | Hydrophilic PCTE | PC | Hydrophilic | 0.01 | N/A |
| | Polyethylene | Polyethylene | Hydrophobic | 0.45 | N/A |
| Bio-rad | PVDF | PVDF | Hydrophobic | 0.2 | N/A |

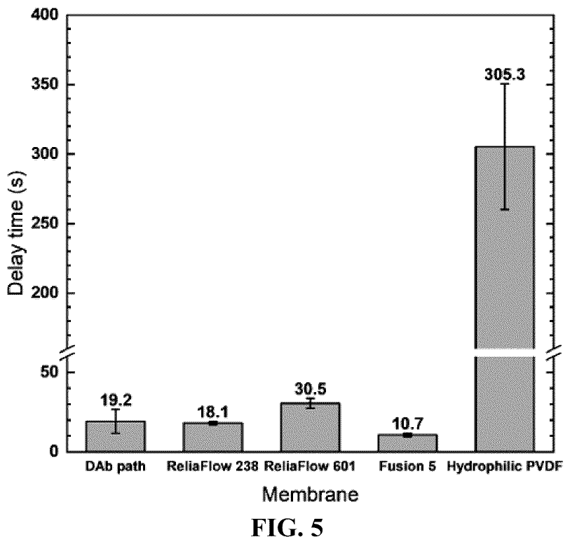

The time delays measured from the sample loading port to the detection window of various membranes are plotted in FIG. 5. Each data point was the average delay time obtained from the tests of seven strips. The results from hydrophilic PCTE, polyethylene, PVDF, and PES membranes were not shown because they could not reach the NC membrane after 30 min from sample loading. Such long delays in PES and PCTE membranes might be caused by the small pore size. The PVDF and polyethylene membranes were unsuitable for this work because their hydrophobic property did not allow the sample liquid to penetrate the membrane. On the other hand, the membranes commonly used for LFA strips showed too rapid flow to be used as a delay pad. Among the tested membranes, the hydrophilic PVDF membrane was the only suitable membrane for attaining the desired delay of 305.3 seconds. Thus, the delay time reaching the NC membrane could be extended by 10 to 30 times longer compared to commonly used membranes for LFA strips. So, the desired 5-minute delay could be secured by using the hydrophilic PVDF membrane as a delay pad. Based on the test results, we concluded that the hydrophilic PVDF membrane was a suitable choice for achieving the flow time difference between the dual paths, which is important in realizing the dual-flow concept in the LFA strip platform.

### Evaluation of Function of Lyophilized CL Substrates

The CL signal and the absorption spectra before and after the lyophilization and reconstitution step were compared to validate the recovered chemical characteristics of the lyophilized CL substrates. For the CL signal evaluation, 200 μl of each CL substrate component was lyophilized in a microcentrifuge tube using the same protocol described above and then reconstituted with 200 μl of the artificial serum. HRP surfaces were prepared using a microfluidic channel-based ELISA plate (Opti 96 plate, MiCo BioMed, South Korea) with the following protocol. Streptavidin-HRP solutions with various dilution factors were incubated for 10 minutes in each microchannel and then followed by three times of wash using 30 μl of phosphate buffered saline (PBS). Finally, the reconstituted CL substrates were introduced into the prepared microfluidic channel-based ELISA plate, and the CL signal was measured using a conventional microplate reader (BioTek Synergy HT, Agilent, USA). For the absorption spectra measurement, Nanodrop Onc^{C} UV-vis spectrometer (ThermoFisher Scientific, USA) was used.

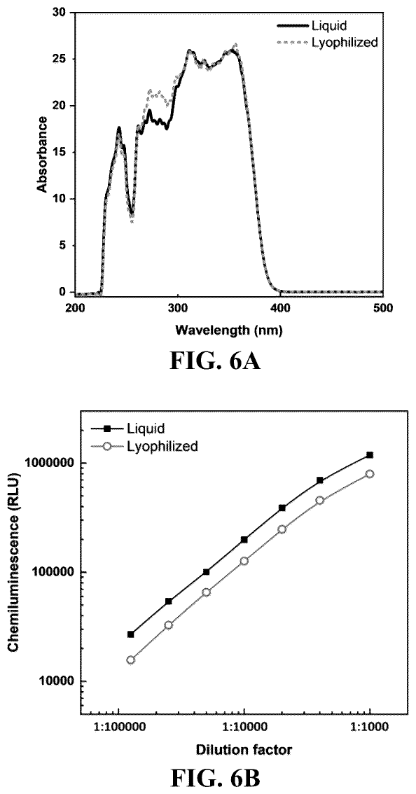

The measured absorption spectra in the ultraviolet and visible light region for the liquid and the lyophilized CL substrate are shown in FIG. 6A. The overlay of the spectra for both the liquid and the reconstituted CL substrates implies that the chemical properties of the CL substrates in liquid format were maintained after the lyophilization and reconstitution of CL substrates using artificial serum as a reconstitution liquid. In addition, FIG. 6B shows the comparison of CL signals from the liquid CL and the lyophilized CL in response to various HRP concentrations on the surface. The CL signal trend from the reconstituted CL substrate was matched well to the signal of the liquid CL substrate and showed a recovery rate greater than 70% in the given concentration range shown in FIG. 6B. Thus, it was successfully demonstrated that the lyophilized CL substrate maintained its capability of generating CL signals after the lyophilization and reconstitution processes and the artificial serum was acceptable for a reconstitution reagent.

### Evaluation of Dual-Flow Lateral Flow Assays for Sequential Dual Flow

FIG. 7 shows the cross-sectional view of a fabricated dual-flow LFA strip in a cartridge. The borders of each membrane are highlighted for better description. Region A shows that both paths for the DAb-HRP and the CL substrate were surely separated by a double-sided tape, and the membranes were contacted well each other. A magnified photograph of the region B shows that the upper strip and lower strip were separately contacted to the NC membrane by the double-sided tape covering beyond the end of the lower strip layer. Thus, the DAb-HRP pad was contacted to the NC membrane directly without any inter-contacts with the lower flow path. Additionally, due to the non-uniform thickness of the dual-flow LFA strip assembly, the overlaid regions in the dual-flow strips were pinched using a specially designed cartridge, as illustrated in FIG. 8. The primary function of the cartridge is to ensure the contact between membranes in the dual flow RDT strips, providing the desired pinch and pressure at the contact regions. So, an extended pressing structure of 200 μm in height and 1 mm in width was designed to press over the overlaid region, which was enough to ensure the contacts but not harm the membranes. In addition, another extruded structure was added to the lid of the cartridge to press and fix the dual-flow LFA strip by fixing the absorbent pad (FIG. 8).

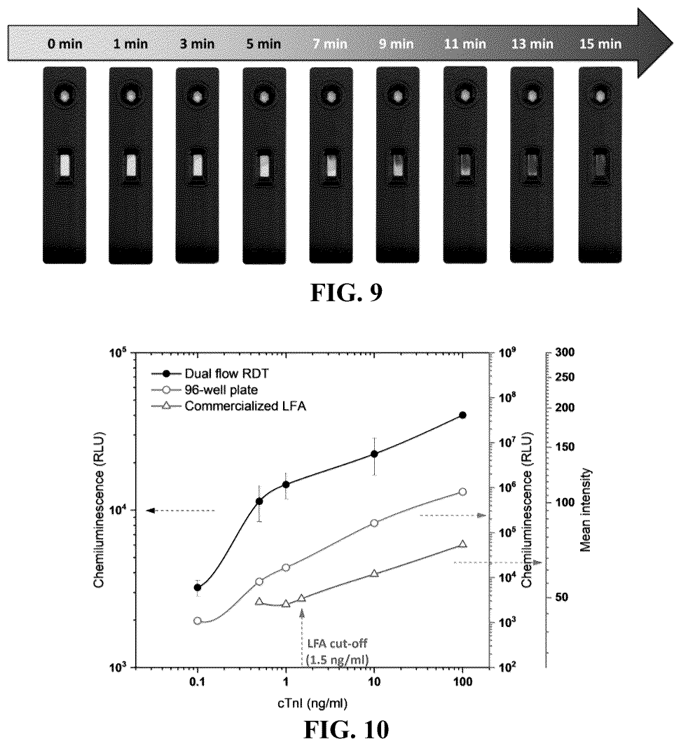

The introduction of sequential flows to the NC membrane was achieved, so the verification of dual flows was performed with the following protocol. The dual-flow LFA strip was prepared to have a light green-colored DAb-HRP in the upper path and a blue-colored CL substrate in the lower path. Then, the dual flow LFA strip was assembled within the 3D-printed cartridge, and an artificial serum of 120 μl was loaded through the sample loading port. Then, the colors appearing on the detection window were observed for 15 minutes to verify the sequential flows of the DAb-HRP and CL substrate. The series of photographs shown in FIG. 9 showed that the observed colors that appeared on the detection window of the NC membrane changed with the elapsing of time. Around 1 minute after the sample loading, the observed color changed to green, indicating that the DAb-HRP reached the NC membrane. 5 to 7 minutes after the sample loading, the observed color changed to a blue color, which indicated the CL substrate reached the detection window. These sequential changes in colors indirectly validated the overall dual-flow concept, which includes the sequential dual-flow control using the delay pad and the pressing structure. The observed color changed to blue entirely after 15 minutes from the sample loading, which corresponds to a very favorable detection time in a point-of-care setting. These results successfully validated the sequential dual-flow design and function.

### Results and Discussion

To compare the assay performance to be obtained from the new RDT platform, the cTnI assay on a 96-well plate was performed first as a standard reference with the following steps: 100 μl of CAb in a concentration of 10 μg/ml was incubated in each well of a 96-well plate at room temperature for 2 hours, and then the wells were washed three times with phosphate buffered saline with 0.1% tween-20 (PBST). A blocking step was followed by dispensing 200 μl of StartingBlock ThermoFisher Scientific, USA) solution into each well, and incubating for 2 hours at room temperature. After completing the blocking step, each well was washed, followed by the incubation step of the target antigen. In detail, 100 μl of artificial serum spiked with various concentrations of human cTnI (30-AT43, Fitzgerald, USA) were dispensed in each well and incubated for 2 hours at room temperature. After the completion of another washing step, DAb-HRP solution in the 1 μg/ml concentration, 100 μl in each well, was dispensed and incubated for 1 hour at room temperature in a dark box. After the final washing step, 100 μl of the CL substrate was dispensed into each well, and then the CL signal was monitored using the BioTek microplate reader. Finally, the cTnI assay was also performed on the CL-based dual-flow RDT platform developed in this work. After 120 μl of cTnI solutions with various concentrations were loaded in the sample loading port of the cartridge, the produced CL signals were measured using the same microplate reader.

The assay performance of the developed new CL-based dual flow RDT platform was compared with the 96-well ELISA as shown in FIG. 10. Each data point is the mean of triplicated results, and the error bar is the standard deviation. For the ELISA performed on the 96-well plate, the error bars are not shown due to its smaller size than the symbol. The assay performance of the new CL-based dual flow RDT platform was reasonably comparable with that of the 96-well ELISA, showing a similar LoD of ˜100 μg/ml. Therefore, the assay performance of the new CL-based dual-flow RDT platform was comparable to the 96-well plate, which allows the new RDT platform to be favorable for the quantitative analysis. For further and practical comparison, we also tested commercially available RDT strips (Lifesign MI TnI, LifeSign LLC, USA) for cInI. Color intensities of the cTnI test line were analyzed using ImageJ software (version 1.54f) and plotted in FIG. 10. The claimed cut-off value is ˜1.5 ng/ml, which matches the results obtained from the commercially available RDT strip kits. Thus, the LoD of the new CL-based dual flow RDT platform for cTnI shows superior performance than the commercially available LFA strip kits.

It is noted that any two quantitative values assigned to a property may constitute a range of that property, and all combinations of ranges formed from all stated quantitative values of a given property are contemplated in this disclosure.

It is noted that one or more of the following claims utilize the term “where” as a transitional phrase. For the purposes of defining the present technology, it is noted that this term is introduced in the claims as an open-ended transitional phrase that is used to introduce a recitation of a series of characteristics of the structure and should be interpreted in like manner as the more commonly used open-ended preamble term “comprising.”

Having described the subject matter of the present disclosure in detail and by reference to specific aspects, it is noted that the various details of such aspects should not be taken to imply that these details are essential components of the aspects. Rather, the claims appended hereto should be taken as the sole representation of the breadth of the present disclosure and the corresponding scope of the various aspects described in this disclosure. Further, it will be apparent that modifications and variations are possible without departing from the scope of the appended claims.

What is claimed is:

1. A lateral flow assay for detecting a target in a liquid sample, the lateral flow assay comprising:
a first flow path and a second flow path for the liquid sample that merge at or before a membrane that generates a signal of immunoassay, wherein:
the first flow path comprises:
a chemiluminescent substrate pad comprising a dried or lyophilized chemiluminescent substrate, wherein the dried or lyophilized chemiluminescent substrate forms a reconstituted chemiluminescent substrate when contacted with the liquid sample;
a delay pad structured and arranged to have a reduced flow velocity; and
the membrane that generates the signal of immunoassay, comprising immobilized capture antibodies; and
the second flow path comprising:
a conjugate pad comprising a dried or lyophilized detection antibody conjugated with an enzyme, wherein the dried or lyophilized detection antibody conjugated with an enzyme forms a reconstituted detection antibody conjugated with an enzyme when contacted with the liquid sample; and
the membrane that generates a signal of immunoassay.

2. The lateral flow assay of claim 1, wherein the lateral flow assay is structured and arranged wherein the liquid sample flowing through the second flow path flows through the membrane that generates the signal of immunoassay before the liquid sample flowing through the first flow path.

3. The lateral flow assay of claim 1, wherein the delay pad comprises a semi-hydrophilic membrane with pore size between 0.1 μm and 1 μm.

4. The lateral flow assay of claim 3, wherein the semi-hydrophilic membrane comprises a PVDF membrane.

5. The lateral flow assay of claim 1, wherein the signal of immunoassay comprises a chemiluminescent signal generated when the reconstituted chemiluminescent substrate reacts with the conjugated enzyme of the reconstituted detection antibody conjugated with an enzyme that is complexed with the immobilized capture antibodies on the membrane that generates the signal of immunoassay.

6. The lateral flow assay of claim 5, wherein the membrane that generates the signal of immunoassay further comprises a control signal.

7. The lateral flow assay of claim 1, wherein the membrane that generates the signal of immunoassay comprises a nitrocellulose membrane.

8. The lateral flow assay of claim 1, further comprising at least one sample pad structured and arranged to receive the liquid sample through absorption, and the liquid sample separately flows through the first flow path and the second flow path.

9. The lateral flow assay of claim 1, wherein:
a first strip defines the first flow path, the first strip comprising:
a first sample pad;
the chemiluminescent substrate pad;
the delay pad; and
the membrane that generates the signal of immunoassay; and
a second strip partially overlapping the first strip defines the second flow path, the second strip comprising:
a second sample pad at least partially overlapping and in fluid communication with the first sample pad; and
the conjugate pad;
wherein the second strip is in fluid communication with the first strip at or before the membrane that generates the signal of immunoassay, at which point the first flow path and the second flow path merge.

10. The lateral flow assay of claim 9, further comprising a separation film positioned between the first strip and the second strip to separate the chemiluminescent substrate pad and delay pad from fluidly communicating with the second strip.

11. The lateral flow assay of claim 10, wherein the separation film comprises a double-sided adhesive tape.

12. The lateral flow assay of claim 9, further comprising a support for the first strip.

13. The lateral flow assay of claim 1, further comprising a wicking pad structured and arranged to absorb excess liquid sample.

14. The lateral flow assay of claim 1, wherein the chemiluminescent substrate pad comprises multiple pads and each pad is partially overlaid with each other, and wherein at least one pad is adjacent to and is at least partially overlaid with the delay pad.

15. The lateral flow assay of claim 1, wherein each pad is at least partially overlaid with an adjacent pad.

16. The lateral flow assay of claim 1, wherein the target is cardiac troponin I, and the dried or lyophilized detection antibody conjugated with an enzyme comprises a dried or lyophilized cardiac troponin I detection antibody conjugated with horseradish peroxidase.

17. A lateral flow assay cartridge comprising:
the lateral flow assay of claim 9, wherein the lateral flow assay further comprises one or more overlaid regions wherein adjacent pads are at least partially overlaid; and
a housing enclosing the lateral flow assay, the housing comprising one or more extended structures aligned with the one or more overlaid regions of the lateral flow assay.

18. The lateral flow assay cartridge of claim 17, wherein the housing comprises a sample loading port positioned over the first sample pad and the second sample pad for receiving the liquid sample, and a detection window positioned over the membrane that generates a signal of immunoassay.

19. A method for detecting a target in a liquid sample using the lateral flow assay of claim 9, wherein the method comprises:
loading a liquid sample onto the first sample pad and the second sample pad;
allowing the liquid sample to flow through the first flow path and the second flow path; and
identifying any immunoassay signal.

20. The method of claim 19, wherein the liquid sample is loaded in a single step.

21. A lateral flow assay for detecting a target in a liquid sample, the lateral flow assay comprising:
a first strip defining a first flow path, the first strip comprising:
a first sample pad;
a chemiluminescent substrate pad adjacent to and in fluid communication with the first sample pad, the chemiluminescent substrate pad comprising a dried or lyophilized chemiluminescent substrate, wherein the dried or lyophilized chemiluminescent substrate forms a reconstituted chemiluminescent substrate when contacted with the liquid sample;
a delay pad adjacent to and in fluid communication with the chemiluminescent substrate pad, the delay pad is structured and arranged to have a reduced flow velocity; and
a membrane that generates a signal of immunoassay adjacent to and in fluid communication with the delay pad comprising immobilized capture antibodies; and
a second strip partially overlapping the first strip and defining a second flow path, the second strip comprising:
a second sample pad at least partially overlapping and in fluid communication with the first sample pad; and
a conjugate pad adjacent to and in fluid communication with the second sample pad, the conjugate pad having an end extending to partially overlap and be in fluid communication with the membrane that generates a signal of immunoassay of the first strip at which point the first flow path and the second flow path merge, and the conjugate pad having a dried or lyophilized detection antibody conjugated with an enzyme, wherein the dried or lyophilized detection antibody conjugated with an enzyme forms a reconstituted detection antibody conjugated with an enzyme when contacted with the liquid sample;
wherein:
the first sample pad and the second sample pad are structured and arranged to receive a liquid sample through absorption, and the liquid sample separately flows through the first flow path and the second flow path; and
the lateral flow assay is structured and arranged wherein the liquid sample flowing through the second flow path flows through the membrane that generates a signal of immunoassay for generating an immunoassay signal before the liquid sample flowing through the first flow path.
